(19) United States
(12) Patent Application Publication
(10) Pub. No.: US 2004/0062729 A1
(43) Pub. Date: Apr. 1, 2004

(54) Photoprotective sunscreen compositions comprising 1,3,5-triazine compounds and triesters of benenetricarboxylic acids

(75) Inventor: Didier Candau, Bievres (FR)

Correspondence Address: BURNS, DOANE, SWECKER & MATHIS, L.L.P., P.O. Box 1404, Alexandria, VA, 22313-1404, US

(73) Assignee: SOCIETE L'OREAL S.A., Paris (FR)

(21) Appl. No.: 10/621,361

(22) Filed: Jul. 18, 2003

Related U.S. Application Data
(63) Continuation of application No. PCT/FR02/00078, filed on Jan. 10, 2002.

(30) Foreign Application Priority Data
Jan. 19, 2001 (FR) 01/00750

(51) Int. Cl. A61K 7/42

(52) U.S. Cl. 424/59

(57) ABSTRACT

Photoprotective cosmetic/dermatological compositions well suited for UV-photoprotecting the skin, lips, hair, eyelashes, eyebrows and/or nails against the damaging effects of UV radiation comprise (i) an effective UV-screening amount of at least one 1,3,5-triazine sunscreen, and (ii) an amount effective to solubilize the totality of said at least one triazine sunscreen (i) of at least one triester of a benzenetricarboxylic acid, formulated into (iii) a topically applicable, cosmetically/dermatologically acceptable carrier therefor.

### CROSS-REFERENCE TO PRIORITY/PCT APPLICATIONS

This application claims priority under 35 U.S.C. § 119 of FR-01/00750, filed Jan. 19, 2001, and is a continuation of PCT/FRO2/00078, filed Jan. 10, 2002 and designating the United States (published in the French language on Jul. 25, 2002 as WO 02/056851 A1; the title and abstract were also published in English), both hereby expressly incorporated by reference.

### BACKGROUND OF THE INVENTION

1. Technical Field of the Invention:

The present invention relates to novel cosmetic or dermatological compositions for topical application, intended in particular for photoprotecting the skin and/or the lips and/or superficial body growths against ultraviolet radiation (compositions called hereinafter more simply antisun or sunscreen compositions), and their uses in numerous cosmetic applications. More precisely still, it relates to compositions with improved photoprotective power comprising, in a cosmetically and/or dermatologically acceptable carrier: (i) at least one 1,3,5-triazine derivative as a lipophilic organic sunscreening agent and (ii) at least one particular, suitably selected oil, which is present in a determined quantity, and which is a triester of a benzenetricarboxylic acid.

2. Description of Background/Related/Prior Art

It is known that light radiation having wavelengths of between 280 nm and 400 nm allows tanning of the human epidermis, and that rays having wavelengths of between 280 nm and 320 nm, known by the name UV-B, cause erythemas and skin burns which can hamper the development of a natural tan; this UV-B radiation should therefore be screened out.

It is also known that UV-A rays having wavelengths of between 320 nm and 400 μm, which cause tanning of the skin, are capable of inducing its impairment, in particular in the case of a sensitive skin or a skin continually exposed to solar radiation. UV-A rays cause in particular a loss of elasticity of the skin and the appearance of wrinkles which lead to premature skin ageing. They promote the onset of the erythematous reaction or amplify this reaction in some subjects and may even be responsible for phototoxic or photoallergic reactions. It is therefore desirable also to screen out UV-A radiation.

Numerous cosmetic compositions intended for the photoprotection (UV-A and/or UV-B) of the skin have been proposed up until now.

These antisun compositions quite often exist in the form of an emulsion of the oil-in-water type (that is to say a cosmetically acceptable carrier consisting of an aqueous dispersing continuous phase and an oily dispersed discontinuous phase) which contains, in various concentrations, one or more lipophilic and/or hydrophilic conventional organic screening agents which are capable of selectively absorbing harmful UV radiation, these screening agents (and their quantities) being selected according to the desired sun protection factor, the sun protection factor (SPF) being mathematically expressed by the ratio of the dose of UV radiation necessary to reach the erythematogenic threshold with the UV-screening agent to the dose of UV radiation necessary to reach the erythematogenic threshold without UV-screening agent.

1,3,5-Triazine derivatives are particularly desirable in antisun cosmetics because they are highly active in UV-B and even in UV-A for some of these compounds according to the nature of the substituents in question. Furthermore, they are photostable, that is to say that they are scarcely or not chemically degraded under the action of UV radiation. They are in particular described in U.S. Pat. No. 4,367,390, EP-863,145, EP-517,104, EP-570,838, EP-796,851, EP-775,698, EP-878,469 and EP-933,376 and the following are known in particular:

2,4,6-tris[p-(2′-ethylhexyl-1′-oxycarbonyl)anilino]-1,3,5-triazine or “ethylhexyl triazone” (INCI name), sold under the trademark “Uvinul T 150” by BASF,

2-[(p-(tert-butylamido)anilino]-4,6-bis-[(p-(2′-ethylhexyl-1′-oxycarbonyl)anilino]1,3,5-triazine or “diethylhexyl butamido triazone” (INCI name) sold under the trademark “UVASORB HEB” by SIGMA 3V,

2,4-bis-{[4-2-ethylhexyloxy)]-2-hydroxy]phenyl}-6-(4-methoxyphenyl)-1,3,5-triazine or “Anisotriazine” (INCI name) sold under the trademark “TINOSORB S” by CIBA SPECIALTY CHEMICALS.

1,3,5-Triazine derivatives have nevertheless the characteristic feature, but also the disadvantage, of being solid at room temperature. As a result, their use in an antisun cosmetic composition involves certain constraints with regard to their formulation and their use, in particular when solvents have to be found which make it possible to properly solubilize them. In this regard, use is today most often made of oils such as esters and more particularly C₁₂-C₅ alkyl benzoates (“FINSOLV TN” from Finetex), or of triglycerides and in particular of triglycerides of C₈-C₁₂ fatty acids (“MIGLYOL 812” from Hüls), or alternatively of monoalcohols or fatty polyols which are oxyethylenated or oxypropylenated (“CETIOL HE” from Henkel or “WITCONOL AM from WITCO). Furthermore, triazine derivatives confer, in the absence of other screening agents, a very limited photoprotective power on these carriers and, in addition, the cosmetic properties attached to them are generally judged to be inadequate.

The present invention proposes a solution for the above problems.

The use of oils of the tribenzoic acid triester type such as tridecyl trimellitate, is well known for the formation of numerous cosmetic products containing a fatty phase. It is described in particular in EP-A-0-792,637 relating to lipsticks. It is also mentioned in EP-A-0-194,055 for the preparation of anhydrous cosmetic formulations free of mineral oil.

In U.S. Pat. No. 4,940,577, this type of oil constitutes the fatty phase of transparent microemulsions of the water-in-oil type with a low water content and necessarily containing, as sole emulsifier, a specific phosphate ester. These microemulsions are used in particular as antisun products based on organic UV-screening agents such as octyldimethyl para-aminobenzoate, octyl cinnamate, octyl salicylate or benzophenone-3 as self-tanning products.

In U.S. Pat. No. 4,940,574, this type of oil is also used as emollient in anhydrous antisun products offering a high degree of protection, containing, in a silicone oil, the combination of two organic UV-B screening agents selected from a cinnamate, a salicylate and a para-aminobenzoate and of one organic UV-A screening agent of the benzophenone type.

### SUMMARY OF THE INVENTION

Following major research in the photoprotection field, it has now unexpectedly and surprisingly been determined that it is possible to substantially improve the photoprotective power of a lipophilic UV-screening agent of the 1,3,5-triazine derivative type contained in photoprotective cosmetic compositions by combining this particular screening agent with a suitably selected specific oil which is a triester of a benzenetricarboxylic acid, it being necessary for this oil to be present in said compositions in a quantity such that, on its own, it allows the entirety of said screening agent to be solubilized by said triester.

This discovery forms the basis of the present invention.

Thus, in accordance with the present invention, novel cosmetic or dermatological compositions are now provided which are characterized in that they comprise, in a cosmetically and/or dermatologically acceptable carrier, (i) at least one UV-screening agent of the 1,3,5-triazine derivative type, and (ii) at least one triester of a benzene-tricarboxylic acid in a sufficient quantity to solubilize all of said screening agent in and of itself.

The present invention also features the use of such compositions in or for the manufacture of compositions intended for protecting the skin and/or the lips and/or superficial body growths such as the hair, the eyelashes, the eyebrows or the nails against the damaging effects of ultraviolet radiation, in particular solar radiation.

Finally, this invention also features the use of at least one triester of a benzenetricarboxylic acid in or for the preparation of a cosmetic composition for protecting the skin and/or the lips and/or superficial body growths such as the hair, the eyelashes, the eyebrows or the nails against ultraviolet radiation, comprising a system which screens UV radiation, containing at least one 1,3,5-triazine derivative, so as to improve the photoprotective power of said composition.

Other characteristics, features, aspects and advantages of the present invention will become apparent from the detailed description which follows.

### DETAILED DESCRIPTION OF BEST MODE AND SPECIFIC/PREFERRED EMBODIMENTS OF THE INVENTION

Among the 1,3,5-triazine derivatives which can be used in the compositions of the present invention, it is possible to use in particular those corresponding to the following formula (I):

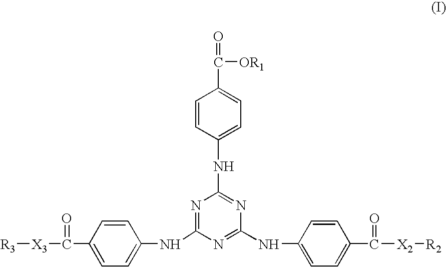

in which:

X₂ and X₃, which may be identical or different, represent oxygen or the radical —NH—;

R₁, R₂ and R₃, which may be identical or different, are selected from: hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a polyoxyethylenated radical comprising from 1 to 6 ethyleneoxide units and whose terminal OH group is methylated; a radical of the following formula (II), (III) or (IV):

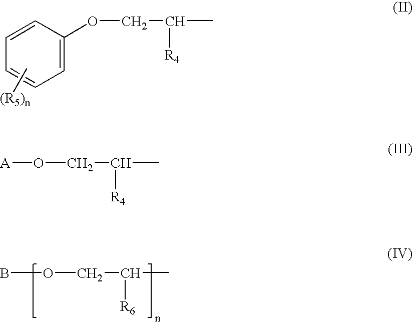

in which:

R₄ is hydrogen or a methyl radical;

R₅ is a C₁-C₉ alkyl radical;

n is an integer ranging from 0 to 3;

m is an integer ranging from 1 to 10;

A is a C₄-C₈ alkyl radical or a C₅-C₈ cycloalkyl radical;

B is selected from: a linear or branched C₁-C₈ alkyl radical; a C₅-C₈ cycloalkyl radical; an aryl radical optionally substituted with one or more C₁-C₄ alkyl radicals;

R₆ is hydrogen or a methyl radical.

A first, more particularly preferred family of 1,3,5-triazine derivatives, which is described in EP-A-0-517,104, is that of the 1,3,5-triazines corresponding to formula (I) above and representing all of the following characteristics:

X₂ and X₃ are identical and represent oxygen;

R₁ is selected from: a C₅-C₁₂ cycloalkyl radical optionally substituted with one or more C₁-C₄ alkyl radicals; a radical of formula (II), (III) or (IV) above in which:

B is a C₁-C₄ alkyl radical;

R₆ is the methyl radical;

R₂ and R₃, which may be identical or different, are selected from: hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (II), (III) or (IV) above in which:

B is a C₁-C₄ alkyl radical;

R₆ is the methyl radical.

A second preferred family of 1,3,5-triazine derivatives according to the invention, which is in particular described in EP-A-0-570,838, is that of the 1,3,5-triazines corresponding to formula (I) and having all of the following characteristics:

X₃ is the radical —NH—;

R₃ is selected from: a linear or branched C₁-C₁₈ alkyl radical; a C₅-C₁₂ cycloalkyl radical optionally substituted with one or more C₁-C₄ alkyl radicals;

R₁ is selected from: hydrogen; an alkali metal; an ammonium radical; a radical of formula (IV); a linear or branched C₁-C₁₈ alkyl radical; a C₅-C₁₂ cycloalkyl radical optionally substituted with one or more C₁-C₄ alkyl radicals;

if X₂ is the radical —NH—, then R₂ is selected from: a linear or branched C₁-C₁₈ alkyl radical; a C₅-C₁₂ cycloalkyl radical optionally substituted with one or more C₁-C₄ alkyl radicals;

if X₂ is oxygen, then R₂ is selected from hydrogen; an alkali metal; an ammonium radical; a radical of formula (IV); a linear or branched C₁-C₁₈ alkyl radical; a C₅-C₁₂ cycloalkyl radical optionally substituted with one or more C₁-C₄ alkyl radicals.

A particularly preferred 1,3,5-triazine of this second family is 2-[(p-(tert-butylamido)anilino]-4,6-bis[(p-(2′-ethylhexyl-1′-oxycarbonyl)anilino]-1,3,5-triazine or “diethylhexyl butamido triazone” sold under the trademark “UVASORB HEB” by SIGMA 3V and corresponds to the following formula:

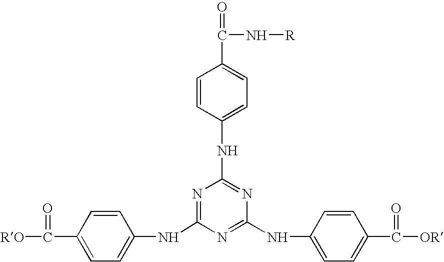

in which R′ is a 2-ethylhexyl radical and R is a tert-butyl radical.

A third preferred family of compounds which can be used in the context of the present invention, and which is described in particular in U.S. Pat. No. 4,724,137, is that of the 1,3,5-triazines corresponding to formula (I) and having all of the following characteristics:

X₂ and X₃ are identical and represent oxygen;

R₁, R₂ and R₃ are identical and represent a C₆-C₁₂ alkyl radical or a polyoxyethylenated radical comprising from 1 to 6 ethyleneoxide units and in which the terminal OH group is methylated.

A particularly preferred 1,3,5-triazine of this third family is 2,4,6-tris[p-(2′-ethylhexyl-1′-oxycarbonyl) anilino]-1,3,5-triazine or “ethylhexyl triazone” sold in particular under the trademark “UVINUL T 150” by BASF and corresponds to the following formula:

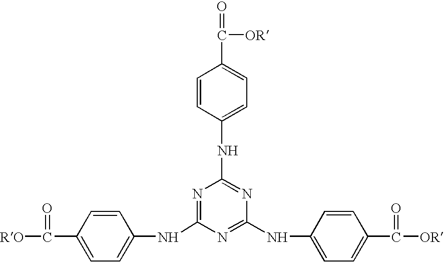

in which R′ is a 2-ethylhexyl radical.

The 1,3,5-triazine derivatives in accordance with the invention may also be selected from the bisresorcinyltriazine derivatives of the following formula (V):

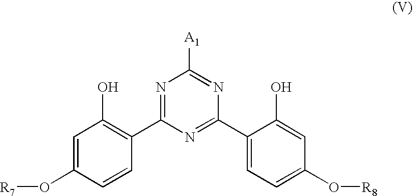

in which:

(i) the radicals R₇ and R₈, which may be identical or different, are each a C₃-C₁₈ alkyl radical; a C₂-C₁₈ alkenyl radical or alternatively a residue of formula —CH₂—CH(OH)—CH₂—OT₁ where T₁ is a hydrogen atom or a C₁-C₈ alkyl radical;

(ii) the radicals R₇ and R₈, which may be identical or different, may also each be a residue of the following formula (1):

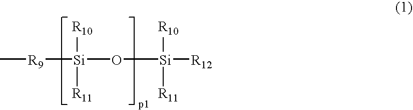

in which:

R₉ is a covalent bond; a linear or branched C₁-C₄ alkyl radical or alternatively a residue of formula —Cₘ₁H₂ₘ₁— or —Cₘ₁H₂ₘ₁—O— where m₁ is a number from 1 to 4;

p₁ is a number from 0 to 5;

the radicals R₁₀, R₁₁, and R₁₂, which may be identical or different, are each a C₁-C₁₈ alkyl radical; a C₁-C₁₈ alkoxy radical or a residue of formula:

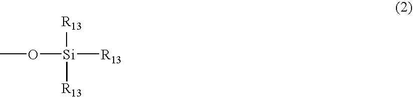

where R₁₃ is a C₁-C₅ alkyl radical;

A₁ is a residue corresponding to one of the following formulae:

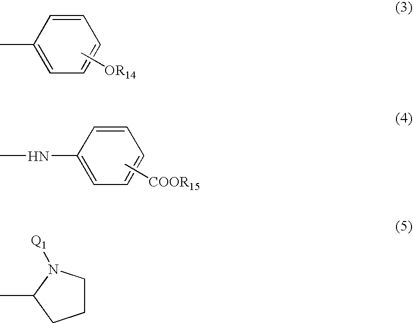

in which:

R₁₄ is a hydrogen atom, a C₁-C₁₀ alkyl radical, a radical of formula: —(CH₂CHR₅—O)ₙ₁R₁₅ where n₁ is a number from 1 to 16, R₁₆ is hydrogen or methyl or alternatively a residue having the structure —CH₂—CH—(OH)—CH₂OT₁ with T₁ having the same meaning as indicated above,

R₁₅ is hydrogen, a metal cation M, a C₁-C₅ alkyl radical or a residue of formula —(CH₂)ₘ₂—OT₁ where m₂ is a number from 1 to 4 and T₁ has the same meaning indicated above,

Q₁ is a C₁-C₁₈ alkyl radical.

In formulae (V) and (1) to (5) described above:

the alkyl radicals are linear or branched and may be selected, for example, from methyl, ethyl, n-propyl, isopropyl, n-butyl, sec-butyl, tert-butyl, amyl, isoamyl, tert-amyl, heptyl, octyl, isooctyl, nonyl, decyl, undecyl, dodecyl, tetradecyl, pentadecyl, hexadecyl, heptadecyl or octadecyl;

the alkenyl radicals may be selected for example from allyl, methallyl, isopropenyl, 2-butenyl, 3-butenyl, isobutenyl, n-penta-2,4-dienyl, 3-methylbut-2-enyl, n-oct-2-enyl, n-dodec-2-enyl, iso-dodecenyl, n-octadec-4-enyl;

the alkoxy radicals are linear or branched and may be selected for example from methoxy, ethoxy, n-propoxy, isopropoxy, n-butoxy, sec-butoxy, tert-butoxy, amyloxy, isoamyloxy or tert-amyloxy;

the C₁-C₅ mono- or dialkylamino radicals may be selected for example from methylamino, ethylamino, propylamino, n-butylamino, sec-butylamino, tert-butylamino, pentylamino, dimethylamino, diethylamino, dibutylamino or methylethylamino;

the metal cations are alkali metal, alkaline earth metal or metal cations selected for example from lithium, potassium, sodium, calcium, magnesium, copper and zinc.

The bisresorcinyltriazine derivatives of formula (V) which can be used according to the invention are screening agents already known per se. They are described and prepared according to the syntheses indicated in particular in EP-A-0-775,698, whose technical content is completely incorporated into the present description.

By way of examples of compounds of formula (V) which can be used, there may be mentioned:

2,4-bis{[4-(2-ethylhexyloxy)-2-hydroxy]phenyl}-6-(4-methoxyphenyl)-1,3,5-triazine;

2,4-bis{[4-(3-(2-propyloxy)-2-hydroxypropyloxy)-2-hydroxy]phenyl}-6-(4-methoxyphenyl)-1,3,5-triazine;

2,4-bis{[4-(2-ethylhexyloxy)-2-hydroxy]phenyl}-6-[4-(2-methoxyethylcarboxyl)phenylamino]-1,3,5-triazine;

2,4-bis{[4-tris(trimethylsiloxysilylpropyloxy)-2-hydroxy]phenyl}-6-(4-methoxyphenyl)-1,3,5-triazine;

2,4-bis{[4-(2″-methylpropenyloxy)-2-hydroxy]phenyl}-6-(4-methoxyphenyl)-1,3,5-triazine;

2,4-bis{[4-(1′,1′,1′,3′,5′,5′,5′-heptamethyl-trisiloxy-2″-methylpropyloxy)-2-hydroxy]phenyl}-6-(4-methoxyphenyl)-1,3,5-triazine;

2,4-bis{[4-(3-(2-propyloxy)-2-hydroxypropyloxy]-2-hydroxy]phenyl}-6-[(4-ethylcarboxyl)phenylamino]-1,3,5-triazine;

2,4-bis{[4-(2-ethylhexyloxy)-2-hydroxy]phenyl}-6-(1-methylpyrrol-2-yl)-1,3,5-triazine.

A compound derived from bisresorcinyltriazine which is more particularly preferred in the context of the present invention is 2,4-bis{[4-2-ethylhexyloxy)]-2-hydroxyl]phenyl}-6-(4-methoxyphenyl)-1,3,5-triazine or “Anisotriazine” sold under the trademark “TINOSORB S” by CIBA SPECIALTY CHEMICALS.

The 1,3,5-triazine derivative(s) are generally present in the compositions of the invention in an amount which may range from 0.5% to 15%, preferably from 1% to 10% by weight, relative to the total weight of the composition.

The triesters of a benzenetricarboxylic acid which can be used in accordance with the present invention are selected in general from the triesters of a benzenetricarboxylic acid with saturated or unsaturated, linear or branched alcohols, having from 3 to 30 carbon atoms, and preferably from 8 to 18 carbon atoms. They correspond to the following general formula (VI):

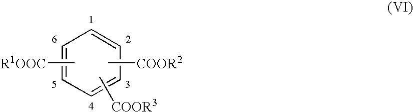

in which R¹, R² and R³, which may be identical or different, are each a saturated or unsaturated, linear or branched hydrocarbon radical having from 3 to 30 carbon atoms and preferably from 8 to 22 carbon atoms, said hydrocarbon radical being still preferably of the alkyl type.

They are selected more preferably from the trimellitic acid esters corresponding to the following general formula (VII):

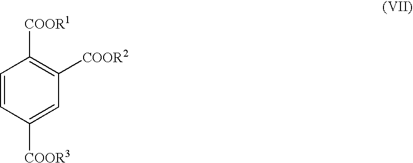

in which R¹, R² and R³ have the same meanings as those indicated for formula (VI) above, and are even still more preferably selected from:

trioctyl trimellitate (R¹=R²=R³=2-ethylhexyl) such as the commercial product sold under the name BISOFLEX TOT by International Speciality Chemical;

triisodecyl trimellitate (R¹=R²=R³=2-butyloctyl) such as the commercial product sold under the name DUB TMI by Stéarineries Dubois or under the name ISOFOL ESTER 1293 by Condéa;

triisohexadecyl trimellitate (R¹=R²=R³=2-hexyldecyl) such as the commercial product sold under the name ISOFOL ESTER 1693 by Condéa;

tricaprilyl/capryl trimellitate (R¹, R² and R³, which may be identical or different are each caprilyl and/or capryl);

tridecyl trimellitate (R¹=R²=R³=—CH₂(CH₂)₁₁—CH₃) such as the commercial product sold under the name DUB TMTD by Stearineries Dubois or the commercial product sold under the name LIPONATE TDTM by LIPO CHEMICALS.

Tridecyl trimellitate is particularly preferred for carrying out the present invention.

The benzenetricarboxylic acid triesters used in accordance with the present invention are generally present in the final compositions in amounts between ranging from 0.1% to 99% by weight relative to the total weight of said composition, and preferably in amounts ranging from 0.5% to 50% by weight.

According to an essential characteristic of the compositions according to the invention, the triester of a benzenetricarboxylic acid should be used in a quantity such that it is sufficient to solubilize, on its own, all, or substantially all, of the screening agent derived from 1,3,5-triazine present in the composition. This minimum quantity of solvent oil intended to bring about complete and stable dissolution of the solid screening agent can be conventionally determined from tests of solubility of said screening agent in this solvent.

The compositions in accordance with the invention may additionally contain other additional organic UV-screening agents, which are active in UVA and/or UVB (absorbers), water-soluble or fat-soluble, or alternatively even insoluble in the cosmetic solvents commonly used in formulation.

The additional organic UV-screening agents are selected in particular from anthranilates; cinnamic derivatives; salicylic derivatives, camphor derivatives; benzophenone derivatives; β,β′-diphenyl acrylate derivatives; benzotriazole derivatives; benzalmalonate derivatives; benzimidazole derivatives; imidazolines; bisbenzoazolyl derivatives as described in EP-669,323 and U.S. Pat. No. 2,463,264; p-aminobenzoic acid (PABA) derivatives; methylenebis(hydroxyphenyl-benzotriazole) derivatives as described in U.S. Pat. Nos. 5,237,071, 5,166,355, GB-2-303,549, DE-197,26,184 and EP-893,119; screening polymers and screening silicones such as those described in particular in WO-93/04665; dimers derived from α-alkylstyrene such as those described in DE-1-9-855,649; 4,4-diarylbutadienes as described in EP-1-008,586, EP-0-967,200 and DE-1-9-755,649; and mixtures thereof.

As examples of additional organic screening agents which are active in UV-A and/or UV-B, there may be mentioned those designated below under their INCI name:

para-Aminobenzoic Acid Derivatives:

PABA,

Ethyl PABA,

Ethyl Dihydroxypropyl PABA,

Ethylhexyl Dimethyl PABA sold in particular under the name “ESCALOL 507” by ISP,

Glyceryl PABA,

PEG-25 PABA sold under the name “UVINUL P25” by BASF,

Salicylic Derivatives:

Homosalate sold under the name “EUSOLEX HMS” by RONA/EM INDUSTRIES,

Ethylhexyl Salicylate sold under the name “NEO HELIOPAN OS” by HAARMANN and REIMER,

Dipropyleneglycol Salicylate sold under the name “DIPSAL” by SCHER,

TEA Salicylate, sold under the name “NEO HELIOPAN TS” by HAARMANN and REIMER,

Cinnamic Derivatives;

Ethylhexyl Methoxycinnamate sold in particular under the trademark “PARSOL MCX” by HOFFMANN LA ROCHE,

Isopropyl Methoxy cinnamate,

Isoamyl Methoxy cinnamate sold under the trademark “NEO HELIOPAN E 1000” by HAARMANN and REIMER,

Cinoxate,

DEA Methoxycinnamate,

Diisopropyl Methylcinnamate,

Glyceryl Ethylhexanoate Dimethoxycinnamate,

β,β′-Diphenylacrylate derivatives:

Octocrylene sold in particular under the trademark “UVINUL N539” by BASF,

Etocrylene, sold in particular under the trademark “UVINUL N35” by BASF,

Benzophenone Derivatives:

Benzophenone-1 sold under the trademark “UVINUL 400” by BASF,

Benzophenone-2 sold under the trademark “UVINUL D50” by BASF,

Benzophenone-3 or Oxybenzone sold under the trademark “UVINUL M40” by BASF,

Benzophenone-4 sold under the trademark “UVINUL MS40” by BASF,

Benzophenone-5,

Benzophenone-6 sold under the trademark “HELIOSORB 11” by NORQUAY,

Benzophenone-8 sold under the trademark “SPECTRA- SORB UV-24” by AMERICAN CYANAMID,

Benzophenone-9 sold under the trademark “UVINUL DS-49” by BASF,

Benzophenone-12,

Benzylidenecamphor Derivative:

3-Benzylidenecamphor manufactured under the name “MEXORYL SD” by CHIMEX,

4-Methylbenzylidenecamphor marketed under the name EUSOLEX 6300 by RONA/EM INDUSTRIES,

Benzylidene Camphor Sulfonic Acid manufactured under the name “MEXORYL SL” by CHIMEX,

Camphor Benzalkonium Methosulfate manufactured under the name “MEXORYL SO” by CHIMEX,

Terephthalylidene Dicamphor Sulfonic Acid manufactured under the name “MEXORYL SX” by CHIMEX,

Polyacrylamidomethyl Benzylidene Camphor manufactured under the name “MEXORYL SW” by CHIMEX,

Benzimidazole Derivatives:

Phenylbenzimidazole Sulfonic Acid sold in particular under the trademark “EUSOLEX 232” by MERCK,

Benzimidazilate sold under the trade trademark “NEO HELIOPAN AP” by HAARMANN and REIMER,

Benzotriazole Derivatives:

Drometrizole Trisiloxane sold under the name “SILATRIZOLE” by RHODIA CHIMIE,

Methylene bis-Benzotriazolyl Tetramethylbutylphenol sold in solid form under the trademark “MIXXIM BB/100” by FAIRMOUNT CHEMICAL or in micronized form as an aqueous dispersion under the trademark “TINOSORB M” by CIBA SPECIALTY CHEMICALS,

Anthranilic Derivatives:

Menthyl anthranilate sold under the trade trademark “NEO HELIOPAN MA” by HAARMANN and REIMER,

Imidazoline Derivatives:

Ethylhexyl Dimethoxybenzylidene Dioxoimidazoline Propionate,

Benzalmalonate Derivatives:

Polyorganosiloxane with a benzalmalonate functional group sold under the trademark “PARSOL SLX” by HOFFMANN LA ROCHE and mixtures thereof.

The additional organic UV-screening agents which are more particularly preferred are selected from the following compounds:

Ethylhexyl Salicylate,

Ethylhexyl Methoxycinnamate,

Octocrylene,

Phenylbenzimidazole Sulfonic Acid,

Benzophenone-3,

Benzophenone-4,

Benzophenone-5,

4-Methylbenzylidene camphor,

Terephthalylidene Dicamphor Sulfonic Acid,

Benzimidazilate,

Methylene bis-Benzotriazolyl Tetramethylbutylphenol,

Drometrizole Trisiloxane and mixtures thereof.

The cosmetic compositions according to the invention may also contain pigments or alternatively nanopigments (mean size of the primary particles: generally between 5 nm and 100 nm, preferably between 10 nm and 50 nm) of coated or uncoated metal oxides such as for example nanopigments of titanium oxide (amorphous or crystallized in rutile and/or anatase form), of iron oxide, of zinc oxide, or zirconium oxide or of cerium oxide which are all UV photoprotective agents well known per se. Conventional coating agents are moreover alumina and/or aluminum stearate. Such nanopigments of metal oxides, coated or uncoated, are described in particular in EP-A-0-518,772 and EP-A-0-518,773.

The compositions according to the invention may also contain agents for artificially bronzing and/or tanning the skin (self-tanning agents) such as for example dihydroxyacetone (DHA).

The compositions of the invention may additionally comprise conventional cosmetic adjuvants, selected in particular from fatty substances, organic solvents other than those specifically used in the context of the present invention, ionic or nonionic thickeners, demulcents, antioxidants, anti-free radical agents, opacifiers, stabilizers, emollients, silicones, α-hydroxy acids, antifoams, moisturizers, vitamins, insect repellents, perfumes, preservatives, surfactants, anti-inflammatory agents, substance P antagonists, fillers, polymers, propellants, alkalinizing or acidifying agents, colorants or any other ingredient normally used in cosmetics, in particular for the manufacture of antisun compositions in the form of emulsions.

The fatty substances may consist of an oil or a wax or mixtures thereof, and they also comprise fatty acids, fatty alcohols and fatty acid esters. The oils may be selected from animal, vegetable, mineral or synthetic oils, and in particular from liquid paraffin, paraffin oil, silicone oils, which are volatile or otherwise, isoparaffins, polyolefins, fluorinated and perfluorinated oils. Likewise, the waxes may be selected from animal, fossil, vegetable, mineral or synthetic waxes which are known per se.

Among the organic solvents other than those in accordance with the invention, there may be mentioned lower alcohols and polyols.

Of course, persons skilled in the art will be careful to choose this or these possible additional compounds and/or their quantities such that the advantageous properties, in particular the increase in the protection factors and the good solubility of the triazine derivatives in the particular solvent oil, which are attached to the compositions in accordance with the invention are not, or not substantially impaired by the addition(s) envisaged.

The compositions of the invention may be prepared according to techniques well known to persons skilled in the art, in particular those intended for the preparation of emulsions of the oil-in-water or water-in-oil type.

These compositions may be provided in particular in the form of a simple or complex (O/W, W/O, O/W/O or W/O/W) emulsion such as a cream, a milk, a gel or a gel cream, of a powder, of a solid stick and optionally may be packaged as an aerosol and may be provided in foam or spray form.

When an emulsion is involved, the aqueous phase thereof may comprise a nonionic vesicular dispersion prepared according to known methods (Bangham, Standish and Watkins, J. Mol. Biol. 13, 238 (1965), FR-2-315,991 and FR-2-416,008).

The cosmetic composition of the invention may be used as a composition for protecting the human epidermis or the hair against ultraviolet rays, as an antisun composition or as a makeup product.

When the cosmetic composition according to the invention is used for protecting the human epidermis against UV rays, or as an antisun composition, it may be provided in the form of a suspension or a dispersion in solvents or fatty substances, in the form of a nonionic vesicular dispersion or alternatively in the form of an emulsion, preferably of the oil-in-water type, such as a cream or a milk, in the form of an ointment, a gel, a gel cream, a solid stick, a powder, a stick, an aerosol foam or a spray.

When the cosmetic composition according to the invention is used for protecting the hair against UV rays, it may be provided in the form of a shampoo, a lotion, a gel, an emulsion, a nonionic vesicular dispersion and may constitute, for example, a rinse-out composition to be applied before or after shampooing, before or after dyeing or bleaching, before, during or after permanent waving or hair straightening, a hair styling or treatment lotion or gel, a blow drying or hair setting lotion or gel, a composition for permanent waving or straightening, dyeing or bleaching the hair.

When the composition is used as a makeup product for the eyelashes, the eyebrows or the skin, such as a treatment cream for the epidermis, a foundation, a lipstick, an eyeshadow, a blusher, a mascara or an eyeliner, it may be provided in an anhydrous or aqueous, solid or pasty form, such as oil-in-water or water-in-oil emulsions, nonionic vesicular dispersions or suspensions.

As a guide, for the antisun formulations in accordance with the invention which have a carrier of the oil-in-water type, the aqueous phase (comprising in particular the hydrophilic screening agents) generally represents from 50% to 95% by weight, preferably from 70% to 90% by weight, relative to the whole formulation, the oily phase (comprising in particular the lipophilic screening agents) from 5% to 50% by weight, preferably from 10% to 30% by weight, relative to the whole formulation, and the (co)emulsifier(s) from 0.5% to 20% by weight, preferably from 2% to 10% by weight, relative to the whole formulation.

As indicated at the beginning of the description, the present invention features the use of a composition as defined above in or for the manufacture of a cosmetic or dermatological composition intended for protecting the skin and/or the hair against ultraviolet radiation, in particular solar radiation.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

The solubilization time of 2,4-bis{[4-2-ethylhexyl-oxy)]-2-hydroxy]phenyl}-6-(4-methoxyphenyl)-1,3,5-triazine (screening agent) in various solvents is determined by means of the following test:

at room temperature, 20 g of solvent are added to 5 g of screening agent,

the mixture thus obtained is heated to 83° C. by means of a water bath,

after cooling, the possible presence of crystals is then observed under a microscope, between a slide and glass coverslips, in polarized light.

The results obtained are reported in the Table I below (the chemical names are given in the CTFA nomenclature, 5th edition, 1993).

TABLE I

| | Storage at | Storage |
|---|---|---|
| | room | in the |
| | temperature | cold (+4° C.) |
| Solvent Oil | for 4 days | for 4 days |
| Apricot stone oil | Crystals | Crystals |
| Dimethyl isosorbide | Crystals | Crystals |
| Isononyl isononanoate | Crystals | Crystals |
| Di(2-ethylhexyl) adipate | Crystals | Crystals |
| Oxypropylenated | Crystals | Crystals |
| butanediol (10 PO) | | |
| Dioctyl malate | Crystals | Crystals |
| 2-Ethylhexyl palmitate | Crystals | Crystals |
| Triglyceride of | Crystals | Crystals |
| capric/caprylic acid (60/40) | | |
| 2-Hexyldecanoic acid | Crystals | Crystals |
| Oxypropylenated dimyristyl | Crystals | Crystals |
| adipate (3 PO) | | |
| Neopentyl glycol | | |
| di-2-ethylhexanoate | Crystals | Crystals |
| Isododecane | Crystals | Crystals |
| Isohexadecane | Crystals/Cloudy | Crystals |
| Cyclopentadimethylsiloxane | Crystals | Crystals |
| Oleyl alcohol | Crystals | Crystals |
| 2-Octyldodecanol | Crystals | Crystals |
| Isodecyl neopentanoate | Crystals | Crystals |
| Isocetyl alcohol | Crystals | Crystals |
| 2-hexyldecanol) | | |
| Oxypropylenated stearyl | Crystals | Crystals |
| alcohol | | |
| Polymethylphenylsiloxane | Crystals | Crystals |
| Glyceryl triisononanoate | Crystals | Crystals |
| Trialkyl citrate | Crystals | Crystals |
| Decyl cocoate | Crystals | Crystals |
| 2-Hexyldecyl octanoate | Crystals | Crystals |
| caprylate | | |
| Oxypropylenated | Crystals | Crystals |
| butanediol (10 PO) | | |
| C₁₂/C₁₅ alcohol benzoate | Crystals | Crystals |
| Copra oil transesterified | Crystals | Crystals |
| with methanol | | |
| Tridecyl trimellitate | Absence of | Absence of |
| (invention) | crystals | crystals |

### EXAMPLE 2

Two antisun formulations (F1, in accordance with the invention; F2, comparative) provided in the form of an oil-in-water type emulsion were prepared by varying the nature of the oil used.

| COMPOSITION | F1 | F2 |
|---|---|---|
| Glyceryl mono/distearate/polyethylene glycol | 1 | 1 |
| stearate (100 EO) mixture ARLACEL 165 FL-ICI) | | |
| Cetyl alcohol | 0.5 | 0.5 |
| Stearic acid from palm oil (STEARINE TP- | 2.5 | 2.5 |
| STEARINERIE DUBOIS) | | |
| Polydimethylsiloxane (DOW CORNING 200 | 0.5 | 0.5 |
| FLUID-DOW CORNING) | | |
| Tridecyl trimellitate (liponate TDTM- | 20 | — |
| LIPO CHEMICALS) | | |
| Triglycerides of caprylic/capric acid | — | 20 |
| (Miglyol 812 Neutral Oil; HÜLS AG) | | |
| 2,4-bis{[4-2-ethylhexyloxy)]-2-hydroxy] | 5 | 5 |
| phenyl}-6-(4-methoxyphenyl)-1,3,5-triazine | | |
| Anisotriazine sold under the trademark | | |
| “TINOSORB S” by CIBA | | |
| Glycerin | 5 | 5 |
| Crosslinked acrylic acid/alkyl acrylate | 1 | 1 |
| copolymer Pemulen TR₁-GOODRICH) | | |
| Hydroxypropyl methyl cellulose (METHOCEL | 0.1 | 0.1 |
| F4M-DOW CHEMICAL) | | |
| Triethanolamine | qs | qs |
| | pH:7 | pH:7 |
| Preservatives | qs | qs |
| Demineralized water qs | 100 g | 100 g |

Each of these emulsions was prepared by dissolving the screening agent in the fatty phase, and then adding the emulsifier to this fatty phase heated to around 80° C., and finally adding, with vigorous stirring, the aqueous phase heated beforehand to this same temperature.

For each of the formulations thus prepared, the sun protection factor (SPF) attached to them was then determined. It was determined using the in vitro method described by B. L. DIFFEY et al. in J. Soc. Cosmet. Chem. 40-127-133 (1989), this method consists in determining the monochromatic protection factors every 5 nm in a range of wavelengths from 290 nm to 400 nm and in calculating therefrom the sun protection factor according to a given mathematical equation.

The results obtained are reported in the following Table II:

TABLE II

| | | SPF in vitro | SPF in vitro | SPF in vitro |
|---|---|---|---|---|
| Formulas | Solvent oil | T = 0 | T = 3 weeks | T = 3 weeks |
| F1 | Tridecyl | 7.8 ± 1.2 | 9.4 ± 1.3 | 8.4 ± 1.2 |
| | trimellitate | | | |
| FII | Miglyol 212 | 4.8 ± 0.9 | 4.9 ± 1.2 | 5.4 ± 1.1 |
| comparative | | | | |

The different results attached to Examples 1 and 2 clearly demonstrate the higher solubilizing power of the tridecyl trimellitate in accordance with the invention and the remarkable beneficial effect provided by the presence of the oil in accordance with the invention with respect to the sun protection factors of the final compositions.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A photoprotective cosmetic/dermatological composition, comprising (i) an effective UV-screening amount of at least one 1,3,5-triazine sunscreen, and (ii) an amount effective to solubilize the totality of said at least one triazine sunscreen (i) of at least one triester of a benzenetricarboxylic acid, formulated into (iii) a topically applicable, cosmetically/dermatologically acceptable carrier therefor.

2. The cosmetic/dermatological composition as defined by claim 1, said at least one 1,3,5-triazine sunscreen having the following formula (I): 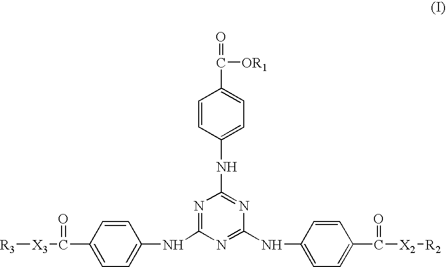
in which X₂ and X₃, which may be identical or different, are each oxygen or —NH—; R₁, R₂ and R₃, which may be identical or different, are each hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a polyoxyethylene radical comprising from 1 to 6 ethyleneoxide structural units and whose terminal OH group is methylated; a radical of the following formula (II), (III) or (IV): 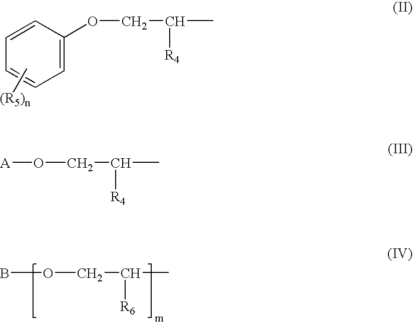
in which R₄ is hydrogen or a methyl radical; R₅ is a C₁-C₉ alkyl radical; n is an integer ranging from 0 to 3; m is an integer ranging from 1 to 10; A is a C₄-C₈ alkyl radical or a C₅-C₈ cycloalkyl radical; B is a linear or branched C ₁-C₈ alkyl radical; a C₅-C₈ cycloalkyl radical; or an aryl radical optionally substituted with one or more C₁-C₄ alkyl radicals; and R₆ is hydrogen or a methyl radical.

3. The cosmetic/dermatological composition as defined by claim 2, wherein said at least one 1,3,5-triazine sunscreen of formula (I), X₂ and X₃ are identical and each is oxygen; R₁ is a C₅-C₁₂ cycloalkyl radical optionally substituted with one or more C₁-C₄ alkyl radicals; or a radical of formula (II), (III) or (IV) in which B is a C₁-C₄ alkyl radical; R₆ is the methyl radical; R₂ and R₃, which may be identical or different, are each hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; or a radical of formula (II), (III) or (IV) in which B is a C₁-C₄ alkyl radical; and R₆ is the methyl radical.

4. The cosmetic/dermatological composition as defined by claim 2, wherein said at least one 1,3,5-triazine sunscreen of formula (I), X₂ and X₃ are identical and each is —NH—; R₃ is a linear or branched C₁-C₁₈ alkyl radical; a C₅-C₁₂ cycloalkyl radical optionally substituted with one or more C₁-C₄ alkyl radicals; R₁ is hydrogen; an alkali metal; an ammonium radical; a radical of formula (IV); a linear or branched C₁-C₁₈ alkyl radical; or a C₅-C₁₂ cycloalkyl radical optionally substituted with one or more C₁-C₄ alkyl radicals; and R₂ is a linear or branched C₁-C₁₈ alkyl radical; or a C₅-C₁₂ cycloalkyl radical optionally substituted with one or more C₁-C₄ alkyl radicals.

5. The cosmetic/dermatological composition as defined by claim 2, wherein said at least one 1,3,5-triazine sunscreen of formula (I), X₂ is oxygen; X₃ is —NH—; R₃ is a linear or branched C₁-C₁₈ alkyl radical; or a C₅-C₁₂ cycloalkyl radical optionally substituted with one or more C₁-C₄ alkyl radicals; R₁ is hydrogen; an alkali metal; an ammonium radical; a radical of formula (IV); a linear or branched C₁-C₁₈ alkyl radical; or a C₅-C₁₂ cycloalkyl radical optionally substituted with one or more C₁-C₄ alkyl radicals; and R₂ is hydrogen; an alkali metal; an ammonium radical; a radical of formula (IV); a linear or branched C₁-C₁₈ alkyl radical; or a C₅-C₁₂ cycloalkyl radical optionally substituted with one or more C₁-C₄ alkyl radicals.

6. The cosmetic/dermatological composition as defined by claim 5, said at least one 1,3,5-triazine sunscreen having the following formula: 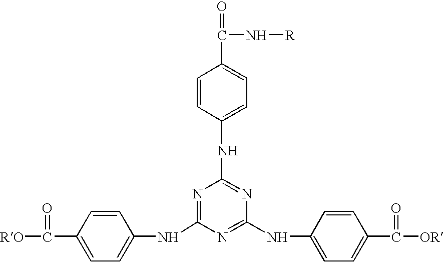
in which R′ is a 2-ethylhexyl radical and R is a tert-butyl radical.

7. The cosmetic/dermatological composition as defined by claim 2, wherein said at least one 1,3,5-triazine sunscreen of formula (I), X₂ and X₃ are identical and each is oxygen; R₁, R₂ and R₃ are identical and each is a C₆-C₁₂ alkyl radical or a polyoxyethylenated radical comprising from 1 to 6 ethyleneoxide units and in which the terminal OH group is methylated.

8. The cosmetic/dermatological composition as defined by claim 7, said at least one 1,3,5-triazine sunscreen having the following formula: 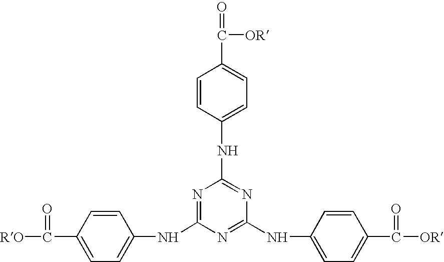
in which R′ is a 2-ethylhexyl radical.

9. The cosmetic/dermatological composition as defined by claim 1, said at least one 1,3,5-triazine sunscreen comprising a bisresorcinyltriazine compound having the following formula (V): 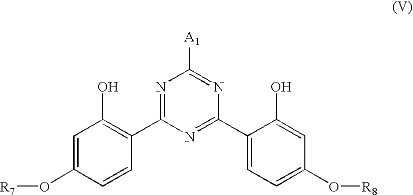
in which (i) the radicals R₇ and R₈, which may be identical or different, are each a C₃-C₁₈ alkyl radical; a C₂-C₁₈ alkenyl radical or a radical of formula —CH₂—CH(OH)—CH₂—OT₁ wherein T₁ is a hydrogen atom or a C₁-C₈ alkyl radical; or (ii) the radicals R₇ and R₈, which may be identical or different, are each a radical of the following formula (1): 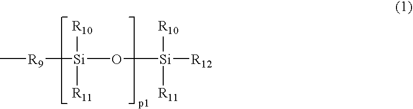
in which R₉ is a covalent bond; a linear or branched C₁-C₄ alkyl radical or a radical of formula —Cₘ₁H₂ₘ₁ or —Cₘ₁H₂ₘ₁—O— wherein m₁ is a number ranging from 1 to 4; p₁ is a number ranging from 0 to 5; the radicals R₁₀, R₁₁, and R₁₂, which may be identical or different, are each a C₁-C₁₈ alkyl radical; a C₁-C₁₈ alkoxy radical or a radical of formula (2): 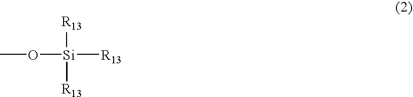
wherein R₁₃ is a C₁-C₅ alkyl radical; A1 is a radical having one of the following formulae (3), (4) and (5): 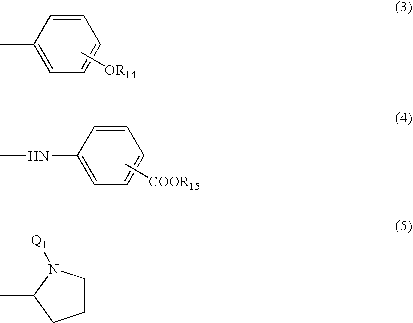
in which R₁₄ is a hydrogen atom, a C₁-C₁₀ alkyl radical, or a radical of formula —(CH₂CHR₅—O)ₙ₁R₁₅ wherein n₁ is a number ranging from 1 to 16; R₁₆ is hydrogen or methyl or a radical having the structure —CH₂—CH—(OH)—CH₂OT₁ wherein T₁ is as defined above; R₁₅ is hydrogen, a metal cation M, a C₁-C₅ alkyl radical or a radical of formula —(CH₂)ₘ₂—OT₁ wherein m₂ is a number ranging from 1 to 4 and T₁ is as defined above; and Q₁ is a C₁-C₁₈ alkyl radical.

10. The cosmetic/dermatological composition as defined by claim 9, said at least one 1,3,5-triazine sunscreen of formula (V) being selected from the group consisting of:
2,4-bis{[4-(2-ethylhexyloxy)-2-hydroxy]phenyl}-6-(4-methoxyphenyl)-1,3,5-triazine;
2,4-bis{[4-(3-(2-propyloxy)-2-hydroxypropyloxy)-2-hydroxy]phenyl}-6-(4-methoxyphenyl)-1,3,5-triazine;
2,4-bis {[4-(2-ethylhexyloxy)-2-hydroxy]phenyl}-6-[4-(2-methoxyethylcarboxyl)phenylamino]-1,3,5-triazine;
2,4-bis{[4-tris(trimethylsiloxysilylpropyloxy)-2-hydroxy]phenyl}-6-(4-methoxyphenyl)-1,3,5-triazine; 2,4-bis{[4-(2″-methylpropenyloxy)-2-hydroxy]phenyl}-6-(4-methoxyphenyl)-1,3,5-triazine;
2,4-bis{[4-(1′,1′,1′, 3′,5′,5′,5′-heptamethyl-trisiloxy-2″-methylpropyloxy)-2-hydroxy]phenyl}-6-(4-methoxyphenyl)-1,3,5-triazine;
2,4-bis{[4-(3-(2-propyloxy)-2-hydroxypropyloxy]-2-hydroxy]phenyl}-6-[(4-ethylcarboxyl)phenylamino]-1,3,5-triazine; and
2,4-bis{[4-(2-ethylhexyloxy)-2-hydroxy]phenyl}-6-(1-methylpyrrol-2-yl)-1,3,5-triazine.

11. The cosmetic/dermatological composition as defined by claim 10, said at least one 1,3,5-triazine sunscreen of formula (V) comprising 2,4-bis{[4-2-ethylhexyloxy)]-2-hydroxy]phenyl}-6-(4-methoxyphenyl)-1,3,5-triazine.

12. The cosmetic/dermatological composition as defined by claim 1, said at least one 1,3,5-triazine sunscreen comprising from 0.5% to 15% by weight, relative to the total weight of the composition.

13. The cosmetic/dermatological composition as defined by claim 1, said at least one 1,3,5-triazine sunscreen comprising from 1% to 10% by weight, relative to the total weight of the composition.

14. The cosmetic/dermatological composition as defined by claim 1, said at least one triester of a benzenetricarboxylic acid having the following structural formula (VI): 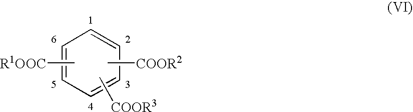
in which R¹, R² and R³, which may be identical or different, are each a saturated or unsaturated, linear or branched hydrocarbon radical having from 3 to 30 carbon atoms.

15. The cosmetic/dermatological composition as defined by claim 14, wherein formula (VI), R¹, R² and R³ have from 8 to 22 carbon atoms.

16. The cosmetic/dermatological composition as defined by claim 14, said at least one triester of a benzenetricarboxylic acid having the following structural general formula (VII): 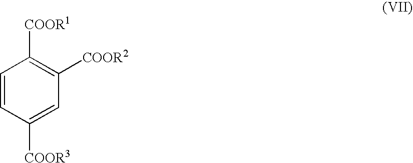
in which R¹, R² and R³ are as defined in formula (VI).

17. The cosmetic/dermatological composition as defined by claim 16, said at least one triester of a benzenetricarboxylic acid being selected from the group consisting of trioctyl trimellitate; tricaprilyl/capryl trimellitate; tridecyl trimellitate; triisododecyl trimellitate and triisohexadecyl trimellitate.

18. The cosmetic/dermatological composition as defined by claim 17, said at least one triester of a benzenetricarboxylic acid comprising tridecyl trimellitate.

19. The cosmetic/dermatological composition as defined by claim 16, said at least one triester of a benzenetricarboxylic acid comprising from 0.1% to 99% by weight, relative to the total weight of said composition.

20. The cosmetic/dermatological composition as defined by claim 16, said at least one triester of a benzenetricarboxylic acid comprising from 0.5% to 50% by weight, relative to the total weight of said composition.

21. The cosmetic/dermatological composition as defined by claim 1, further comprising at least one other organic screening agent active in the UV-A and/or UV-B range.

22. The cosmetic/dermatological composition as defined by claim 21, said at least one other organic UV-screening agent being selected from the group consisting of anthranilates; cinnamic derivatives; salicylic derivatives, camphor derivatives; benzophenone derivatives; β,β′-diphenyl acrylate derivatives; benzotriazole derivatives; benzalmalonate derivatives; benzimidazole derivatives; imidazolines; bisbenzoazolyl derivatives; p-aminobenzoic acid (PABA) derivatives; methylenebis(hydroxyphenylbenzotriazole) derivatives; screening polymers and screening silicones; and dimers derived from α-alkylstyrene.

23. The cosmetic/dermatological composition as defined by claim 22, said at least one other organic UV screening agent being selected from among the following compounds:
Ethylhexyl Salicylate,
Ethylhexyl Methoxycinnamate,
Octocrylene,
Phenylbenzimidazole Sulfonic Acid,
Benzophenone-3,
Benzophenone-4,
Benzophenone-5,
4-Methylbenzylidene Camphor,
Terephthalylidene Dicamphor Sulfonic Acid,
Benzimidazilate,
Methylene bis-Benzotriazolyl Tetramethylbutylphenol,
Drometrizole Trisiloxane, and mixtures thereof.

24. The cosmetic/dermatological composition as defined by claim 1, further comprising coated or uncoated pigments or nanopigments of metal oxides.

25. The cosmetic/dermatological composition as defined by claim 24, said pigments or nanopigments comprising titanium, zinc, iron, zirconium or cerium oxides, or mixtures thereof.

26. The cosmetic/dermatological composition as defined by claim 1, further comprising at least one agent for artificially bronzing and/or tanning the skin.

27. The cosmetic/dermatological composition as defined by claim 1, further comprising at least one adjuvant selected from the group consisting of fatty substances, organic solvents, ionic or nonionic thickeners, demulcents, antioxidants, anti-free radical agents, opacifiers, stabilizers, emollients, silicones, α-hydroxy acids, antifoams, moisturizers, vitamins, insect repellents, perfumes, preservatives, surfactants, anti-inflammatory agents, substance P antagonists, fillers, polymers, propellants, alkalinizing or acidifying agents, colorants.

28. The cosmetic/dermatological composition as defined by claim 1, formulated as a nonionic vesicular dispersion, an emulsion, a cream, a milk, a gel, a gel cream, a suspension, a dispersion, a powder, a solid stick, a foam or a spray.

29. The cosmetic/dermatological composition as defined by claim 1, formulated as a makeup for the eyelashes, the eyebrows or the skin and comprising an anhydrous or aqueous, solid or paste, or an emulsion, a suspension or a dispersion.

30. The cosmetic/dermatological composition as defined by claim 1, formulated for protecting the hair against ultraviolet rays and comprising a shampoo, a lotion, a gel, an emulsion, or a nonionic vesicular dispersion.

31. A regime or regimen for UV-photoprotecting the skin, lips, hair, eyelashes, eyebrows and/or nails against the damaging effects of UV radiation, comprising topically applying thereon an effective amount of a photoprotective cosmetic/dermatological composition which comprises (i) an effective UV-screening amount of at least one 1,3,5-triazine sunscreen, and (ii) an amount effective to solubilize the totality of said at least one triazine sunscreen (i) of at least one triester of a benzenetricarboxylic acid, formulated into (iii) a topically applicable, cosmetically/dermatologically acceptable carrier therefor.

32. A method for improving the photoprotective capability of at least one 1,3,5-triazine sunscreen, comprising formulating therewith at least one triester of a benzenetricarboxylic acid.
